(19) United States
(12) Patent Application Publication
(10) Pub. No.: US 2025/0001419 A1
(43) Pub. Date: Jan. 2, 2025

(54) Preloaded light impermeable testing apparatus containing optical property-modifying additives for testing rFC-reactive substances, methods of use, and methods of making

(71) Applicant: Caitlin Rose EAST, Atlanta, GA (US)

(72) Inventor: Caitlin Rose EAST, Atlanta, GA (US)

(21) Appl. No.: 18/203,039

(22) Filed: May 29, 2023

(51) Int. Cl. B01L 3/00; G01N 21/64

(52) U.S. Cl. CPC B01L 3/5085; G01N 21/6428; B01L 2200/12; B01L 2200/16; B01L 2300/0829; B01L 2300/12; B01L 2300/16; G01N 2021/6439

(57) ABSTRACT

A preloaded light impermeable testing apparatus containing optical property-modifying additives for detecting an rFC-reactive substance, wherein at least a portion of said testing apparatus has been preloaded with at least one solution comprising an rFC reagent and/or at least one rFC-reactive standard. Methods of use of the testing apparatus are disclosed. Methods of depositing reagents comprising test reagents on a testing apparatus are also disclosed.

### BACKGROUND OF THE INVENTION

Endotoxin is the lipopolysaccharide (LPS) component of the bacterial wall of gram-negative bacteria. The human immune system responds to the presence of LPS, and LPS has a significant impact on human health. LPS activates the immune system and causes fever. LPS has three distinct parts: the O-antigen, core, and lipid A. The lipid A portion (lipid component) is associated with the toxicity of the molecule. Lipid A triggers an immune response resulting in fever, diarrhea, and sepsis. LPS is present in intact living gram negative bacteria and in bacterial wall fragments. LPS requires high temperature to degrade, and sterilization cycles are not sufficient for removal. As such, a specific test for endotoxin is necessary for final release of medical devices, parenteral, and intrathecal drugs.

Historically, the pharmaceutical industry used rabbits to test for endotoxin. The rabbit pyrogen test (RPT) is a biological test used for endotoxin testing. In RPT, a small amount of sample (drug product or water from device extraction) is injected into the ear vein of a rabbit. At predetermined time intervals after the injection, the rabbit's body temperature is measured to detect any signs of fever. If the rabbit develops a fever, the sample is considered to contain endotoxin.

The use of RPT in pharmaceutical testing is a topic of ethical concern, since the testing can cause significant pain and distress to the rabbits involved. Ethical considerations aside, the RPT is not quantitative and can result in false positives. Since the introduction of the Limulus amebocyte lysate (LAL) assay by Levin and Bang, in the 1970s, the pharmaceutical and medical device industry has relied on Limulus polyphemus (commonly known as the Atlantic horseshoe crab or American horseshoe crab), and Tachypleus tridentatus (commonly known as the Chinese horseshoe crab, Japanese horseshoe crab, or tri-spine horseshoe crab) to support the final release of life-saving drugs and devices. The LAL assay utilizes a protein called Factor C, which is present in the amebocytes (blood cells) of the horseshoe crab. In the LAL assay, Factor C recognizes the presence of endotoxins in a sample and triggers a cascade reaction, leading to the formation of a clot (gel-like substance) or the appearance of a yellow color; LAL assays quantify the concentration of LPS through absorbance detection methods. The degree of clotting or color development is directly proportional to the concentration of endotoxins in the sample. LAL is sourced from animals, so it remains a topic of ethical discussions, but it does provide highly reproducible, quantitative results.

The industry has conflicting approaches to the collection of blood from horseshoe crabs. Regionally, Limulus polyphemus are fished for and returned. Tachypleus tridentatus are fished for and bled to a fatal conclusion. Concerns for the sustainability of this method and reliance on this resource are rising as the population of the world continues to increase, along with the increasing need for testing. Global access to vaccines is increasing, with advanced cell and gene therapies becoming increasingly available, the demand for LAL will outpace the availability of raw materials. Furthermore, risks to supply due to climate factors should be considered.

Moreover, as climate change progresses, horseshoe crabs are likely to be negatively affected. Horseshoe crabs have survived for over 455 million years, but they are vulnerable to changes in their environment. Horseshoe crabs use sandy beaches for nesting during breeding season, and coastal erosion and rising sea levels can reduce the availability of suitable habitats. Consequently, their population could decline, and the genetic diversity of the species could be negatively impacted.

Genetic diversity is essential for the adaptation and evolution of species to changing environmental conditions. Reduced genetic diversity can lead to decreased fitness, reduced reproductive success, and increased susceptibility to disease. The production of Factor C protein is horseshoe crabs is genetically controlled, and the industry observes performance variations between vendors. Each vendor fishes in their geographic region, and the resulting reagents are products from geographically disparate populations.

If genetic diversity is impacted due to climate change, the production of Factor C could be affected, which would impact the performance and sensitivity of the LAL assay.

Even if Factor C is unaffected, reduced genetic diversity could lead to inbreeding depression, which is the loss of fitness or increased susceptibility to disease that accompanies increased rates of inbreeding. Ultimately this results in reduced survival, decreased reproductive success, and a decline in overall population size. Reduced population size would negatively affect the industry's ability to keep up with increased global demand.

To assuage these concerns, there are recombinant alternatives available; recombinant Factor C (rFC) is a recombinant formulation of the LAL assay. rFC is a synthetic version of the naturally occurring Factor C protein. When rFC encounters LPS, it initiates a biochemical reaction that ultimately leads to the activation of a proenzyme, which generates a fluorescent signal or a color change; fluorescence is the detection mode of commercially available rFC assays. Recombinant methods provide a means for controlling the manufacture of Factor C and reduce inter-batch variability. However, the recombinant assays are just as time intensive as traditional assay types.

Recombinant formulations of reagents present a unique opportunity to improve the sensitivity and specificity of the test method. The promise of alternative assay types is due to the detection method employed; fluorescence-based assays detect at the single-photon level, and the heightened theoretical sensitivity exceeds the sensitivity of absorbance-based assays approved by the United States Pharmacopeia (USP), European Pharmacopoeia (EP), and Japanese Pharmacopeia (JP). Traditional assays, the LAL assays: gel clot, kinetic chromogenic, and turbidimetric assay, are all absorbance-based assays. These assays require many manual steps and have limited sensitivity.

Current assay modalities in use by pharmaceutical and medical device manufacturers use amebocyte lysate or a recombinant form of Factor C, the clotting protein in the amebocyte. These assays work by creating a standard curve with a known amount of endotoxin and measuring the resulting optical changes in color intensity, turbidity, or fluorescent signal output (chromogenic method, turbidimetric method, and recombinant Factor C, respectively).

In routine use, the assay is performed using test tubes for gel clot preparation or a 96-well microplate for turbidimetric or chromogenic assay types. All three of these reagent chemistries rely on the reconstitution of assay reagents and involve many manual pipetting steps.

When performing a conventional assay, a skilled analyst must manually reconstitute the lyophilized reagents with endotoxin-free water, without introducing additional endotoxin into the solution. When working with a 96-well microplate format, an analyst is preparing 21 samples in duplicate with PPCs, and a 5-point standard curve. To prepare this assay, over 200 pipetting steps are performed. The pipetting process is prone to contamination with endotoxins, which are common and persistent in the environment.

Using a standard 96-well microplate assay also requires 100 μL of sample per well (400 μL per test when in duplicate with PPCs), which is not an issue for many product types, but it is significant when testing autologous cell therapy products that have a final production volume of less than 10 mL.

In recombinant forms of the assay, reagents may be available in lyophilized and liquid form. Liquid reagents can be easier to work with but have a shorter shelf life. Liquid reagents eliminate some pipetting steps, but there are multiple reagent components to mix (Factor C, buffer, fluorogenic substrate) which adds an equivalent number of pipetting steps, negating the reduced pipetting effort required as compared to lyophilized traditional reagents.

The conventional LAL assay and recombinant forms both use a Control Standard Endotoxin (CSE) for standard controls, as required by the various compendia, or the gold standard, Reference Standard Endotoxin (RSE). CSE is an alternative to RSE. Since different endotoxin moieties have varying immunological impacts, the industry uses Endotoxin Units (EU) as a measure of biological activity. Each LAL batch varies due to the biological nature of the product and differences in manufacturing processes. To standardize the results across vendors and chemistries when using CSE, each lot of LAL is matched with CSE, and the reconstitution volume is determined so that the assay performance is equivalent to LAL and RSE. The reconstitution volume of the CSE when matched with a particular lot of LAL only corresponds to that lot of LAL, as the biological activity of each batch of material varies, and the effective potency of each batch of CSE also varies. It is important when using CSE that the correct reconstitution volume is used; otherwise, the results will be inaccurate. Furthermore, reagent batches of both recombinant products and traditional LAL products will have different reconstitution volumes with different batches of CSE. If the wrong CSE is used with a product batch, the results are invalid without additional testing, causing delays in product release.

Absorbance, electronically, is the promotion of an electron from one state, usually ground, to a higher, less stable state. The ground state of the material is the lowest possible energy configuration. When matter is in a state that is higher in energy (less stable) than the ground state, it is referred to as “excited”, sometimes denoted by an asterisk. When using absorbance to determine the concentration of endotoxin in a sample, it is important that the substrate or testing apparatus for the assay is clear, so that the light will pass through the reaction vessel to the solution without interference. If a colored reaction vessel (containing optical property-modifying additives) is used, the light emitted from the solution will be absorbed, reflected, or scattered, which interferes with the accuracy of the measurements.

Absorbance is an easy, but non-specific method for measuring concentration. Absorbance-based assays utilize Beer's law to obtain the concentration of the substance of interest. In the case of conventional LAL assays, it would be the chromogenic reaction substrate, which is read at 405 nm by a spectrophotometer, or generalized light scattering measured at 340 nm, for the turbidimetric assay. These assays take around 1 hour and rely on kinetic measurements to track the rate of change of signal development. The faster the signal develops, the more endotoxin is in the reaction vessel.

Currently available solutions to solve the issues associated with LAL assays support absorbance-based detection methods.

A plate reader, also known as a microplate reader or multiwell plate reader, is a scientific instrument commonly used in biomedical and life science research laboratories. It is designed to analyze multiple samples simultaneously in microplates, which are typically flat trays with multiple wells arranged in a grid pattern.

Clear or white microplates, using titanium dioxide additives, are poor substrate choices for fluorescence-based assays, and only fluorescence-based assays have the sensitivity required to detect low levels of endotoxin in products that have low yield volumetrically, or require significant dilution to overcome interference due to the sample matrix.

Clear microplates are not an appropriate choice for fluorescence-based assays because they allow for a significant amount of background fluorescence to be detected by a plate reader. The additional fluorescence signal interferes with the accuracy of the assay by increasing the signal to noise ratio and decreasing the dynamic range of the assay. The background noise impacts signal reliability more when endotoxin concentrations are low, which is usually the case for products being tested by GMP (Good Manufacturing Practice) facilities. Signal is even lower when the sample volume used is reduced.

In a fluorescence assay, a fluorescent molecule is used to label a specific molecule of interest. In the case of recombinant Factor C assays, the fluorogenic substrate is activated by the recombinant Factor C protein when it is bound to an endotoxin molecule. When excited by the 380 nm wavelength of light, the fluorogenic substrate emits light at a longer wavelength of 440 nm. The amount of light emitted corresponds to the amount of endotoxin present in the sample.

In a clear microplate, the excitation light can pass through the bottom of the microplate and reflect off the surface, causing background fluorescence. This background fluorescence can contribute to the total fluorescence signal, leading to inaccurate measurements and false positives.

Other options include using white microplates, which are polystyrene with titanium dioxide additives to create an opaque, white, highly reflective surface. White microplates reflect light, which can increase the background fluorescence and make it more difficult to distinguish the signal from the background. The reflective properties of white microplates can cause an uneven distribution of light across the wells, leading to variability in signal intensity and reduced assay precision.

Crosstalk occurs when using a microplate and signal from an adjacent well interferes with the measurement of the target well. Crosstalk is the unintentional transfer of signal from one well to another due to signal bleed through or overlapping detection areas. This can lead to false negatives or false positives, and high percent CVs. Crosstalk is reduced by optimizing experiment design including reaction vessel selection. Crosstalk occurs in clear microplates and commercially available solutions.

Methods and devices have been developed to reduce the number of steps or automate the steps in absorbance-based endotoxin testing. Some methods include automating one or more pipetting or aliquoting steps, automated mixing of samples, or preloading reagents and rFC-reactive standards or PPCs in testing substrates for absorbance-based assays.

Existing solutions designed to reduce reagent consumption focus on absorbance-based testing and utilize highly proprietary detection equipment. These devices produce supply chain risks, as they work with a single vendor's reagent or single vendor's detection equipment. In the event of a reagent shortage, the manufacturers utilizing this validated solution cannot release life-saving drugs. Any delay in release for cell therapy drugs can result in patient death due to reduced efficacy of therapies. Recipients of cell therapy are already in critical states, and even a day delay can impact product performance or viability of cells and is unacceptable.

Additionally, cell therapy products are derived from living cells and can pose a risk to technicians handling the final product samples during testing. Based on individual product composition, a biosafety cabinet may be required for sample handling and processing, so it is important that equipment and supplies can be used in a biosafety cabinet, if necessary.

Other solutions use robotic liquid handlers to measure and distribute samples and reagents in a microplate. Once prepared, the microplate is loaded into the reader with a robotic manipulator or manually loaded by a technician. These systems are expensive and require daily maintenance to perform consistently. The environmental conditions of the laboratory must be tightly controlled, as these systems are very sensitive to humidity and electrostatic discharge. Contamination can be an issue with these systems, and due to the number of variables, troubleshooting performance issues requires many runs to determine the root cause of variability or failures.

Liquid handlers would need additional customizations to be compliant with cell therapy products' Biosafety Level requirements.

All developed methods or devices, are missing one or more of the following aspects, low-cost automation designed into testing apparatus, disposable to prevent contamination, acceptable for use as a standard or alternative method under the compendia, built in test method validation, designed to maximize fluorescence signal to noise ratio, compatible with existing testing equipment, compatible with multiple reagent vendors, compatible with multiple lot of reagents, simple to operate, reduces sample volume, or reduces the reagent requirement. Accordingly, there is a need for a semi-automated light impermeable testing apparatus containing optical property-modifying additives or procedure for testing and analyzing the endotoxin concentration in a liquid sample designed for fluorescence measurements which reduces or eliminates potential for error and is acceptable for use under the compendia as a standard or alternative method.

### SUMMARY OF THE INVENTION

Accordingly, light impermeable testing apparatuses containing optical property-modifying additives and methods are disclosed wherein the number of test steps are reduced significantly, thereby minimizing contamination, repetitive stress injuries, and errors, and thus improving accuracy. The methods are suitable for use with recombinant Factor C products utilizing fluorogenic substrates for signal production. The disclosed light impermeable testing apparatuses containing optical property-modifying additives and measurement methods are suitable for use in pharmaceutical and biopharmaceutical manufacturing and are compatible with European Pharmacopeia and Japanese. Pharmacopeia Bacterial Endotoxin Test (BET) and global equivalent pharmacopeia BET standards. The disclosed light impermeable testing apparatus containing optical property-modifying additives are suitable for use in the United States as an alternative method to LAL as approved by the FDA. For medical device manufacturing, the disclosed embodiments are compatible with endotoxin regulations and standards found in the international pharmacopeia and consensus standards organizations and global equivalent standards.

Embodiments of the invention improve the recombinant Factor C (rFC) fluorescence-based BET by the creation of specialized light impermeable testing apparatuses containing optical property-modifying additives with detection reagents (may be recombinant endotoxin detection reagents and/or rFC-reactive standards) preloaded onto said testing apparatus. As used herein, additives mean a substance used in the manufacturing process of the light impermeable testing apparatus that are effective light absorbers and scatter light across a range of wavelengths, including visible and ultraviolet. These additives will appear black to the human eye. One such additive is carbon black; carbon black particles absorb and scatter light. The apparatus may contain one optical window without additives. In one embodiment, a preloaded light impermeable testing apparatus containing optical property-modifying additives contains additives is disclosed wherein the preloaded light impermeable testing apparatus containing optical property-modifying additives has been preloaded with at least one fluorescence-based detection reagent and/or at least one rFC-reactive standard. These preloaded light impermeable testing apparatuses containing optical property-modifying additives may be used in tests for determining the concentration of rFC-reactive substances in an aqueous sample. As used herein rFC-reactive substances means a substance that binds to recombinant Factor C. Examples of rFC-reactive substances include endotoxin but not 1,3-β-D-glucans. rFC-reactive standards comprise rFC-reactive substances therein. The present invention may be used with any commercial source of fluorescence-based detection reagents. Suitable detection reagents for detecting rFC-reactive substances include recombinant Factor C reagents that use fluorogenic substrates and any other reagents capable of reacting with LPS to produce a measurable fluorescence signal.

The present invention may reduce the number of steps the user has to perform in preparing and measuring both the calibration standards and samples. This may reduce the need for a high level of skill, experience, and training; and reduces costs, time, and the opportunity for human error. In addition, embodiments of the invention may be configured or utilized in a manner that complies with compendia requirements and FDA regulations.

Embodiments of the invention are also suitable for use with all quantitative fluorescence-based methods relating the output of light to the quantity of endotoxin in aqueous solution. These detection reagents may utilize endpoint or kinetic measurements, but all will use fluorescence-based detection methods.

In another embodiment, at least a portion of the preloaded light impermeable testing apparatus containing optical property-modifying additives may have a modified surface. The surface may be modified by plasma treatment or corona treatment. Alternatively, the surface may be modified using at least one coating. The coating may be a static coating, a dynamic coating or combinations thereof. Suitable static coatings include but are not limited to, polyethylene glycol (PEG), collagen, and combinations thereof. Suitable dynamic coatings include but are not limited to polyethylene glycol (PEG), sodium deoxycholate, and combinations thereof.

In yet another embodiment, the preloaded light impermeable testing apparatus containing optical property-modifying additives may have a mechanical barrier between at least one of the portions. The mechanical barrier may be soluble. Suitable soluble coatings include but are not limited to, 1-3-β-D-glucans such as laminarin or curdlan. The preloaded light impermeable testing apparatus containing optical property-modifying additives may further comprise a portion identification mechanism, such as a tracer. In another embodiment, the preloaded light impermeable testing apparatus containing optical property-modifying additives may be a microplate. In yet another embodiment, the preloaded light impermeable testing apparatus containing optical property-modifying additives may have a barrier material to protect the preloaded light impermeable testing apparatus containing optical property-modifying additives from environmental exposure and surface contamination.

In another embodiment, a method for measuring an rFC-reactive substance in a sample is disclosed. The method comprises contacting the sample with a preloaded light impermeable testing apparatus containing optical property-modifying additives wherein at least a portion of the preloaded light impermeable testing apparatus containing optical property-modifying additives has been preloaded with at least one reagent comprising a detection reagent (components may include, but are not limited to enzyme, buffer, and fluorogenic substrate) and/or at least one rFC-reactive standard, thereby making a prepared sample. The fluorescence of the sample may then be measured.

In another method embodiment, at least a portion of the preloaded light impermeable testing apparatus containing optical property-modifying additives may have a modified surface. The surface may be modified by plasma treatment or corona treatment. Alternatively, the surface may be modified using at least one coating. The coating may be a static coating, a dynamic coating or combinations thereof. Suitable static coatings include but are not limited to, polyethylene glycol (PEG), collagen, and combinations thereof. Suitable dynamic coatings include but are not limited to polyethylene glycol (PEG), sodium deoxycholate, and combinations thereof.

In yet another embodiment, the preloaded light impermeable testing apparatus containing optical property-modifying additives may be a microplate. In yet another embodiment, the preloaded light impermeable testing apparatus containing optical property-modifying additives may have a barrier material to protect the preloaded light impermeable testing apparatus containing optical property-modifying additives from environmental exposure and surface contamination.

In another embodiment, a method for depositing at least one detection reagent on a microplate containing optical property-modifying additives is disclosed. Test reagents be any reagent that aids in testing samples. Suitable detection reagents include fluorescence-based detection reagents and rFC-reactive standards. Suitable detection reagents are described above and may comprise recombinant Factor C, fluorogenic substrate, and buffer. rFC-reactive standards include Control Standard Endotoxin (CSE) and USP Endotoxin Reference Standard (RSE). The method may comprise providing a light impermeable testing apparatus containing optical property-modifying additives having a well array comprising of a plurality of wells, wherein each well has at least one optical window surface and plurality of non-optical window surfaces. A first liquid solution having at least one component of fluorescence-based detection reagent may be placed on a first non-optical window surface of at least one well. The first liquid solution may be dried on the first non-optical window surface thereby depositing the component comprising a detection reagent on the first non-optical surface to form a preloaded light impermeable testing apparatus containing optical property-modifying additives.

In another method embodiment, lyophilized beads comprising rFC-reactive standards and/or at least one component comprising a detection reagent may be used to preload the light impermeable testing apparatus containing optical property-modifying additives with the respective components described previously.

In another embodiment, the method for depositing at least one detection reagent on a light impermeable testing apparatus containing optical property-modifying additives may further comprise placing a second solution having at least one rFC-reactive standard therein on a second non-optical window surface. The second liquid solution may be dried on the second non-optical window surface thereby depositing the rFC-reactive standard on the second non optical window surface.

In another embodiment, the light impermeable testing apparatus containing optical property-modifying additives may be a microplate. In yet another embodiment, the method may further comprise covering the preloaded light impermeable testing apparatus containing optical property-modifying additives with a barrier material after the drying step to protect the preloaded light impermeable testing apparatus containing optical property-modifying additives from environmental exposure and surface contamination.

### BRIEF DESCRIPTION OF THE DRAWINGS

FIG. 1 shows one embodiment of the invention wherein a component comprising a detection reagent or standard may be deposited on the sidewalls of a microplate while the microplate is positioned on a surface at an angle between 0 and 90 degrees.

FIG. 2 shows one embodiment of the invention where the testing apparatus comprises detachable microwell strips of standard dimensions.

FIG. 3 shows one embodiment of the invention where the testing apparatus comprises detachable microwell strips of reduced vertical height and total volume.

FIG. 4 shows one embodiment of the invention where the testing apparatus utilizes the same well spacing (center to center) as a standard 96-well microplate but features wells with half the diameter of a standard 96-well microplate.

FIG. 5 shows one embodiment of the invention where a multichannel pipettor is loading reagent onto the testing apparatus on a flat surface.

### DETAILED DESCRIPTION OF THE INVENTION

In one embodiment, a preloaded light impermeable testing apparatus containing optical property-modifying additives is disclosed wherein the preloaded light impermeable testing apparatus containing optical property-modifying additives has been preloaded with at least one component comprising a detection reagent and/or at least one rFC-reactive standard. The preloaded light impermeable testing apparatus containing optical property-modifying additives is designed to measure the concentration of bacterial endotoxin in samples. It may also be used to provide calibration data from known spikes using an rFC-reactive standard. The preloaded light impermeable testing apparatus containing optical property-modifying additives may be designed to satisfying all the current BET pharmaceutical regulation requirements and may be used with rFC reagents. The rFC-reactive standards may be endotoxin that has been calibrated to the control standard endotoxin (CSE) and the regulatory standard endotoxin (RSE). Where other methods are acceptable or have been validated as being equivalent and acceptable to regulatory agencies, a stored calibration based on historical data can be used instead of the results from individual standards.

Accordingly, preloaded light impermeable testing apparatuses containing optical property-modifying additives are disclosed wherein the number of testing steps are reduced significantly, thereby minimizing contamination, timing delays, and mismatches, thus improving accuracy. The methods are suitable for use with fluorescence-based detection methods. The methods may be used with a standard fluorescence microplate reader with built in thermal control, a mixer, and a fluorescence detector or photomultiplier tube to determine BET results.

In another embodiment, the rFC-reactive standard may be preloaded in at least three different portions of the preloaded light impermeable testing apparatus containing optical property-modifying additives. These three different portions may form a calibration portion. The concentration of the rFC-reactive standard in each portion may be the same or different. If endotoxin is used, the first portion may have an amount such that when an aqueous sample (or blank water) is present in that portion, the endotoxin concentration in the sample ranges from 0.005 to 0.5 Endotoxin Units per milliliter (EU/mL). Similarly, the second portion may have an amount corresponding to a concentration ranging from 0.05 to 5.0 EU/mL and the third portion may have an amount corresponding to a concentration ranging from 0.5 to 50 EU/mL.

In another embodiment, a portion of the light impermeable testing apparatus containing optical property-modifying additives will contain additives and the remaining portion will not contain additives. This embodiment may for an alternative method validation to compare rFC reagents with LAL BET reagents. The entire light impermeable testing apparatus containing optical property-modifying additives will contain preloaded rFC-reactive standards and may contain either or both rFC reagents loaded in the perpendicular surface of the region of the light impermeable testing apparatus containing optical property-modifying additives and LAL reagents on the perpendicular surface of the region of the testing apparatus without additives.

In another embodiment, at least two portions of the preloaded light impermeable testing apparatus containing optical property-modifying additives may form a sample measurement portion. The two portions may be loaded with an rFC-reactive standard to form spikes.

The detection reagent and/or rFC-reactive standard may be deposited onto various light impermeable testing apparatuses containing optical property-modifying additives, such as onto the sidewalls of a microplate well to allow a sample blank measurement, onto the optical window of a microplate well, onto a soluble coating, or onto an optically translucent or reflective insoluble film. Alternatively, the test reagents may be added as lyophilized beads or particles, or deposited into a carrier media that is added to the light impermeable testing apparatus containing optical property-modifying additives.

In another embodiment, at least a portion of the preloaded light impermeable testing apparatus containing optical property-modifying additives may have a modified surface. The surface may be modified using plasma etching. Alternatively, the surface may be modified using at least one coating. The coating may be a static coating, a dynamic coating, or a combination thereof. Suitable static coatings include but are not limited to polyethylene glycol (PEG), collagen, and combinations thereof. Suitable dynamic coatings include but are not limited to polyethylene glycol (PEG), sodium deoxycholate, and combinations thereof.

In yet another embodiment, the preloaded light impermeable testing apparatus containing optical property-modifying additives may have at least one mechanical barrier between at least one of the portions. The mechanical barrier may be soluble.

The light impermeable testing apparatus containing optical property-modifying additives with preloaded reagents or rFC-reactive materials may be packaged such that it is sealed from the environment by using a material that prevents moisture, bacteria, and endotoxin agents from contaminating the preloaded reagents. Accordingly, in yet another embodiment, the preloaded light impermeable testing apparatus containing optical property-modifying additives may have a barrier material to protect the preloaded light impermeable testing apparatus containing optical property-modifying additives from environmental exposure, light exposure, and surface contamination. In another embodiment, the preloaded light impermeable testing apparatus containing optical property-modifying additives may be a microplate. In another embodiment, the preloaded light impermeable testing apparatus containing optical property-modifying additives may be a single reaction vessel.

Sample introduction errors may be further reduced by a plurality of optional identification mechanisms on the preloaded light impermeable testing apparatus containing optical property-modifying additives or in a reader configured to read or measure samples in the light impermeable testing apparatus containing optical property-modifying additives. The identification mechanisms may identify the sample to the user or notify the user if additional reagents are required. Suitable identification means may include optical markers such as colorimetric markers, alphanumeric markers, or light emitting diodes. In one embodiment, the identification mechanism may be a tracer. A tracer is an inert compound that is added to a fluid to aid in determining the volume, fluid location and movement (fluid motions). The tracer may also be used to aid in validating the measurement data. Suitable tracers include but are not limited to fluorescent dyes.

In another embodiment a method for measuring endotoxin in a sample is disclosed. As used in this specification, the term “sample” may include not only the sample to be analyzed, but water that shows no reaction with the detection reagent or recombinant product employed. Samples of non-reactive water may also be referred to as “LAL reagent water”, “Water for BET”, or “Water for injection” (WFI). The method may comprise contacting the sample with a preloaded light impermeable testing apparatus containing optical property-modifying additives, wherein at least a portion of the preloaded light impermeable testing apparatus containing optical property-modifying additives has been preloaded with at least one component comprising a detection reagent and/or at least one rFC-reactive standard thereby making a prepared sample. A fluorescence measurement of the sample may be then taken.

The sample may contact more than one portion of the light impermeable testing apparatus containing optical property-modifying additives. The prepared sample contacting the preloaded light impermeable testing apparatus containing optical property-modifying additives may or may not come in to contact with a detection reagent. For example, the prepared sample may be a blank or negative control that does not contact any detection reagent or only comes into contact with a detection reagent. In another method, a portion of the substrate, maybe further preloaded with at least one rFC-reactive standard. If the rFC-reactive standard is an endotoxin standard, it may be present in a plurality of concentrations, wherein each concentration is present on a different portion of the light impermeable testing apparatus containing optical property-modifying additives, as described above. It may also be present in a plurality of volumes, wherein each volume is present on a different portion of the light impermeable testing apparatus containing optical property-modifying additives as described above. The endotoxins may be preloaded onto the light impermeable testing apparatus containing optical property-modifying additives containing additive such that a standard curve may be generated as required in USP 85. In another embodiment, the plurality of rFC-reactive standard concentrations or volumes may be used to generate a standard curve.

In another method embodiment, at least a portion of the preloaded light impermeable testing apparatus containing optical property-modifying additives may have a modified surface. The surface may be modified using plasma etching. Alternatively, the surface may be modified using at least one coating. The coating may be a static coating, and dynamic coating or combination thereof suitable static coatings include but are not limited to polyethylene glycol (PEG), collagen, and combinations thereof. Suitable dynamic coatings include, but are not limited to polyethylene glycol, sodium deoxycholate, and combinations thereof.

In yet another embodiment, the preloaded light impermeable testing apparatus containing optical property-modifying additives may be a microplate. In yet another embodiment, the preloaded light impermeable testing apparatus containing optical property-modifying additives, may have a barrier material to protect the preloaded light impermeable testing apparatus containing optical property-modifying additives from environmental exposure and surface contamination.

In another embodiment, a method for depositing at least one component comprising a detection reagent on a microplate containing optical property-modifying additives is disclosed. Components of detection reagents may be any material that comprises a solution that aids in testing samples using fluorescence-based detection. Suitable components of detection reagents include, but are not limited to recombinant Factor C, buffer, enzyme and fluorogenic substrate. Suitable detection reagents are described above and may comprise rFC reagents. rFC-reactive standards are also described above and include a USP Endotoxin reference standard (RSE), that has been calibrated to the current World Health Organization International Standard for Endotoxin. The method may comprise providing a light impermeable testing apparatus containing optical property-modifying additives, having a well array comprising a plurality of wells wherein each well may have at least one optical window surface and a plurality of non-optical window surfaces. The additive will modify the optical properties of the material comprising the light impermeable testing apparatus containing optical property-modifying additives. The additive shall absorb light across a wide range of wave lengths in the visible spectrum. Suitable additives include but are not limited to carbon black. A first liquid solution having at least one component comprising a detection reagent therein may be placed on a first non-optical window surface of at least one well. The first solution may be dried on the first non-optical window surface, thereby depositing at least one component comprising a detection reagent on the first non-optical surface to form a preloaded light impermeable testing apparatus containing optical property-modifying additives. The rFC-reactive standard may be placed in a plurality of concentrations, we are in each concentration is present in a separate well of the light impermeable testing apparatus containing optical property-modifying additives. In another embodiment, the rFC-reactive standard may be placed in a plurality of volumes, we are in each volume is present in a separate well of the light impermeable testing apparatus containing optical property-modifying additives.

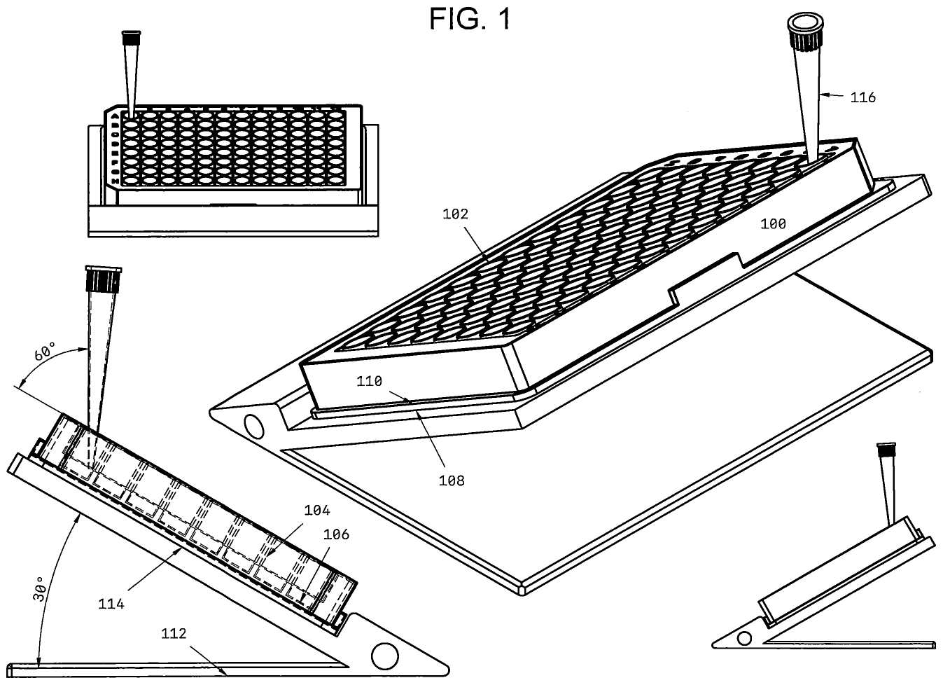

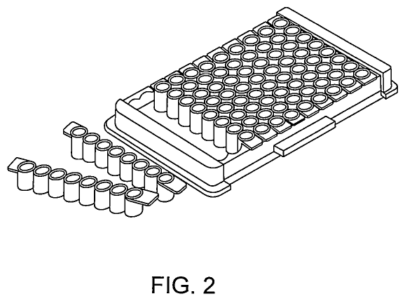

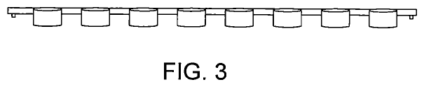

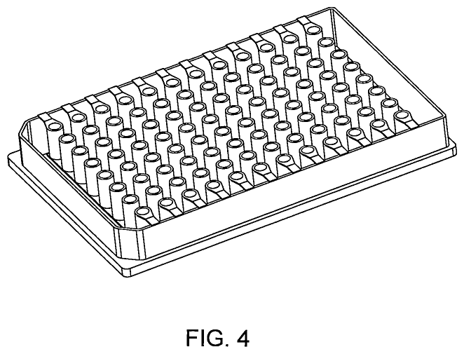

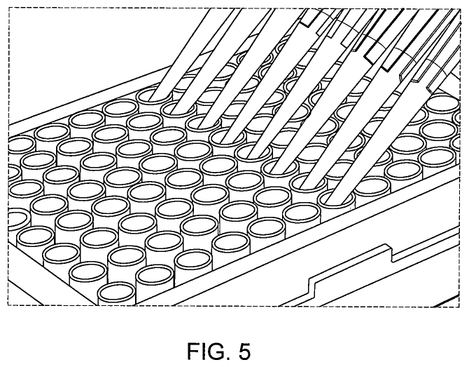

In another embodiment, the light impermeable testing apparatus containing optical property-modifying additives may be a microplate. In yet another embodiment, the method may further comprise covering the preloaded light impermeable testing apparatus containing optical property-modifying additives with a barrier material after the drying step to protect the preloaded light impermeable testing apparatus containing optical property-modifying additives from environmental exposure and surface contamination. Suitable light impermeable testing apparatus containing optical property-modifying additives include any testing apparatus that aids in evaluating or testing a sample, such as microplates containing optical property-modifying additives available from Nunc. As shown in FIG. 1, the microplate (100) may have multiple sample wells arranged in a 2×3 rectangular matrix. Microplates typically have 6, 24, 96, 384, or 1536-wells. In one environment, the microplate may have 96-wells (102). Although the holding capacity of individuals within the microplate is usually the same, the holding capacity of the wells may vary from microplate to microplate. The columns of the wells may be removable, see FIG. 2. See FIG. 3, where the volume of the well is greatly reduced by reducing the depth. See FIG. 4, where are the volume is reduced by reducing the radius of the well. In another embodiment, the shape of the well is such that the total volume of sample within the well is greatly reduced. The sidewalls (104) and bottoms (106) of the wells (102) may be curved or straight, such of the wells are semi spherical, cylindrical or rectangular in shape. The microplate may also comprise a substantially planar bottom surface (108) such that the microplate rest flat on working surfaces, as shown in FIG. 5. Working surfaces may include but are not limited to the ground lab bench tops, microplate readers, and heating plates as well as manufacturing surfaces, such as tables, conveyors and rollers. FIG. 1 shows a fabricated angled platform providing a flat surface for the microplate to rest on at an angle between 0 and 90 degrees. It is also possible that the microplate does not rest on a working surface at all, but is suspended above the working surface via a suspension means such as hooks, clips, etc. The microplate containing optical property-modifying additives may be made of a variety of materials, including polystyrene, and polypropylene or polycarbonate. An optical detection microplate may be made with polystyrene or other suitable polymer that does not interfere with the chemical performance of the detection reagents with the sample. Carbon black or similar shall be added to the material to aid in fluorescence-based detection methods.

One or more portions of the light impermeable testing apparatus containing optical property-modifying additives may have modified surfaces. The portions with modified surfaces may include but are not limited to the sidewalls and wells. The surfaces may be modified by any means known to those of ordinary skill in the art, including but not limited to, applying a coating, radiation, plasma etching, UV light and ozone, or dissolved reagents which may dynamically cover the surface, so that the interaction of the surfaces and reagents or samples mimic that of standard microplate analysis so that the manufacturer's specifications or compendia standards for analysis are met.

In one embodiment the surface of the light impermeable testing apparatus containing optical property-modifying additives may be modified to control the biochemical rFC and rFC-reactive substance interaction or to control the surface energy. Controlling the level of the surface chemical interaction with the reaction chemistries may improve the repeatability and accuracy of the biochemical performance. For example, material suitable for manufacturing the light impermeable testing apparatus containing optical property-modifying additives may also biochemically inhibit or enhance the rFC or rFC-reactive substance reaction chemistry. This biochemical interaction between the material surface and the reaction chemistries may be controlled or reduced with the application of a coating or through a chemical modification of the surface. Additionally, the unmodified surface of the light impermeable testing apparatus containing optical property-modifying additives may have an undesirable surface energy for the microfluidics present on the light impermeable testing apparatus containing optical property-modifying additives. The surface energy may also be modified to a desired value through chemical modification or the addition of a coating to make the surface energy more hydrophilic or more hydrophobic, or to achieve any other surface energy between these states. By optimizing the surface energy, the microfluidics present on the light impermeable testing apparatus containing optical property-modifying additives may also be optimized.

Another means to modify light impermeable testing apparatus containing optical property-modifying additives surfaces include plasma etching, where the surface is modified by having it exposed to plasma to affect a particular final surface chemical structure. Different elements may be added to the plasma to modify the chemistry of the surface, for example, oxygen or ammonia. Additional means include the use of permanent static or dynamic surface coatings. Static surface coatings may be added to form a layer on the light impermeable testing apparatus containing optical property-modifying additives surface to change the surface character. Static surface coatings may be applied as a solution with a solvent and dried or applied by surface grafting wherein the coating is chemically bonded to the surface. Examples of static coatings that may be grafted or applied as a coating include but are not limited to polyethylene glycol (PEG) and collagen. Dynamic surface coatings may be added to the reagents, samples, or standards and coat the surface in situ as fluids move on the light impermeable testing apparatus containing optical property-modifying additives or in sample wells. Examples of dynamic coatings include but are not limited to PEG and surfactants like sodium deoxycholate.

In one embodiment, the method may comprise providing a microplate containing optical property-modifying additives, having a well array comprising a plurality of wells, wherein each well has at least one optical window surface and a plurality of non-optical window surfaces; providing a liquid solution having at least one component comprising a detection reagent therein; placing the liquid solution on a first of said non-optical window surfaces of at least one well; and drying the liquid solution on the first non-optical window surface, thereby depositing at least one component of the detection reagent on the first non-optical window surface. In another embodiment, the deposition steps may be repeated on subsequent non-optical window surfaces. Such detection reagents are deposited on subsequent sidewall portions.

The components of the detection reagents may be added to non-optical window surfaces of the well, to allow an initial optical measurement of the sample before the detection reagents have had a chance to mix. This is useful for determining the optical zero, or baseline optical measurement. In addition, each component comprising a detection reagent has an optical signature that may be used to check that the correct levels of detection reagents are added, prior to the reaction beginning. In another embodiment, each detection reagent may be tagged with an optical or fluorescent material that is inert to the endotoxin test reaction. If the user finds incorrect levels of the expected levels of detection reagents prior to the reaction taking place (reaction lag phase period), then the user may reject the measurement test for that sample. This is of great value to the pharmaceutical user, as any Out of Specification (OOS) test must be evaluated and explained.

In another method embodiment, the method may comprise providing a liquid solution having at least one component comprising a detection reagent therein; providing a microplate containing optical property-modifying additives, having a substantially planar bottom surface, a plurality of edges (110), and a well array, where the bottom surface is angled (FIG. 1) with respect to a horizontal working surface (112), and wherein the well array complex comprises, a plurality of wells, having a plurality of sidewalls. A first of the plurality of edges (110) may be tilted such that the first edge is inclined (114) with respect to the horizontal working surface, such that the bottom surface is no longer substantially parallel with respect to the working surface and such that a side wall portion closest to the working surface is in a generally parallel orientation with respect to the working surface. The liquid solution (116) may be placed on the intersection of the bottom face of the well and side wall portion, and then dried thereby depositing the testing region on the intersection of the well face and sidewall portion. Any means suitable to transfer a liquid may be suitable, including but not limited to a pipette, or a spray nozzle.

Any drying process is suitable for the present invention, as long as the drying process does not alter the reactivity of the detection reagents. These drying processes include but are not limited to a vacuum drying process at ambient temperature or a freeze-drying process (lyophilization). In yet another embodiment, the liquid solution may be dried at ambient temperature or freeze dried. It should be understood that the liquid solution need not be dried completely; it may be partially dried, especially if non-aqueous solvents are used. It is sufficient that the detection reagent is physically immobilized after it is deposited such that it remains in place. There may be some liquid still present after the detection reagent is immobilized if a glycerin paste is used, as in certain pharmaceuticals and other materials prepared for stable storage. The same process may be used with both round-walled and flat-walled wells. The tilt-position of the microplate may be maintained during the deposition steps through the use of a supporting means such as a stand or brace (FIG. 1).

In another embodiment, the microplate may be rotated, and the deposition steps may be repeated with subsequent edges of the microplate such that additional detection reagents are deposited on subsequent sidewall portions. In another method embodiment, at least one component comprising a detection reagent may be present in every well. In another method embodiment, at least one component compromises an rFC-reactive standard. In yet another method embodiment, the rFC-reactive standard may be present in a plurality of concentrations, wherein each concentration is present in a different well. In yet another method embodiment the same concentration of rFC-reactive standards may be present and deposited in a plurality of volumes. In yet another method embodiment, the microplate may further comprise a well identification mechanism.

Many approaches to the detection reagent deposition may be used to reduce mixing time, bubble formation, resolubilization time, ease of manufacturing, and detection sensitivity. The approaches may encompass both chemical and physical means to produce the desired results. Chemical means may include the use of chemical additives. Examples of chemical additives include solubility enhancing agents, such as the saccharides sucrose, glucose, and mannitol, as well as anti-flaking agents, such as aqueous polymer solutions comprising poly(ethylene oxide), hydroxypropyl cellulose, or hydroxypropyl methyl cellulose, or agents designed to prevent degradation such as dextran and various saccharides such as lactose and trehalose. Physical means may include various coating, spraying, or drying techniques during the deposition process. It is important to note that the light impermeable testing apparatus containing optical property-modifying additives for suitability with fluorescence-based detection reagents will not react with any 1,3-β-D-glucan-containing products, making all these chemical means appropriate choices for inert additives that will not impact testing outcomes. Traditional LAL reagents do react to 1,3-β-D-glucan-containing compounds, such as cellulose and sugars.

In some embodiments, at least one component comprising a detection reagent may be deposited in every well. Alternatively, there is no detection reagent in any of the wells, allowing the user to add detection reagent from a preferred supplier. In one embodiment the detection reagent may be recombinant Factor C. The use of rFC, or other fluorescence-based non-rFC reactive tracers to recombinant Factor C and endotoxin may be used to reduce errors.

The rFC reactive standard, maybe deposited only a portion of the wells. In addition, various wells may be preloaded or pre-deposited with rFC-reactive standard with different concentrations of the rFC reactive substance therein such that the user merely has to add the sample to be tested to the wells. The rFC-reactive standard may be deposited in only a portion of the wells. In addition, various wells may be preloaded or pre-deposited with rFC-reactive standard with different concentrations of the rFC-reactive substance therein, such that the user merely has to add the sample to be tested to the wells. In one embodiment, the detection reagent and rFC-reactive substance may be deposited in the wells such that all the tests and replicates required by the USP Chapter 85: Bacterial Endotoxins Test General Chapter (USP 85) may be performed simply by adding the samples. In such an embodiment, each well comprises either a separate given test, or a replicate of a given test. In one embodiment, the lowest concentration may be confirmed in four replicates, wherein 4 of the 96 wells each comprise one replicate. Alternatively, the wells may be preloaded with rFC-reactive standards such that the inhibition/enhancement tests (or “spikes”), including replicates, may be performed. Alternatively, the wells may be preloaded such that the quantitative tests, wherein the concentration of bacterial endotoxins in a given sample is quantified, may be performed. In yet another embodiment, the wells may be preloaded such that all the tests and replicates required under USP 85, including the assay sensitivity, the inhibition/enhancement, and quantitative tests, may be performed on the same microplate. Similar concepts may be employed with any light impermeable testing apparatus containing optical property-modifying additives or any portion of a light impermeable testing apparatus containing optical property-modifying additives and are not limited to microplates with wells.

In one embodiment, the wells may be covered with a seal means, such as an adhesive label with adhesive only on the portions of the label outside the well opening. The seal means may be made of a barrier material that prevents the passage of water and oxygen, whereby the wells may be kept dry to a humidity level less than about 5%.

The disclosed methods may be used to pre-deposit rFC reagents or rFC reactive standards in pre-cleaned (endotoxin free) 96 or 384-well microplates. The detection reagents, in a liquid solution, may be placed on the walls of the wells, or on the optical window surface of the optical well. The liquid solution may also comprise chemical additives such as solubility enhancing agents and anti-flaking agents. The disclosed methods allow the reagents to be deposited on the walls of the standard 96 or 384-well microplates without interfering with the optical window or the optical path, thereby allowing an initial sample fluorescence measurement.

In another embodiment, a light impermeable testing apparatus containing optical property-modifying additives is disclosed wherein at least a portion of the light impermeable testing apparatus containing optical property-modifying additives has been preloaded with at least one component comprising an rFC detection reagent. The light impermeable testing apparatus containing optical property-modifying additives is suitable for fluorescence monitoring of liquids and use in performing rFC assays for endotoxins.

Reagents for the rFC assays may be isolated in segments of the light impermeable testing apparatus containing optical property-modifying additives. The light impermeable testing apparatus containing optical property-modifying additives may be disposable. The light impermeable testing apparatus containing optical property-modifying additives may have a variety of forms, geometries and shapes, including a typical microplate shape. Other suitable forms include, but are not limited to cards, cartridges, or discs. The light impermeable testing apparatus containing optical property-modifying additives may also be configured such that samples and fluids may be added to it. The light impermeable testing apparatus containing optical property-modifying additives also allows for mixing of samples as the light impermeable testing apparatus containing optical property-modifying additives is shaken, swirled, spun or rotated. The light impermeable testing apparatus containing optical property-modifying additives also allows for the optical monitoring of fluorescence signals in liquids.

The light impermeable testing apparatus containing optical property-modifying additives can be used for performing analytical functions including, but not limited to, measurement of samples with an added positive product control that is an rFC-reactive substance, such as endotoxin, or glucan spike, measurement of water blanks (free of endotoxin or rFC reagent), measurement of a series of at least three calibration solutions. Moreover, the light impermeable testing apparatus containing optical property-modifying additives may be used for performing all the analytical functions listed in two or more duplicates.

The light impermeable testing apparatus containing optical property-modifying additives may be used with an optical apparatus or reader that measures the times between optical fluorescence states or the optical fluorescence change between times. The preloaded light impermeable testing apparatus containing optical property-modifying additives may also be used for confirmation that the reagents and analyzer meet specifications, calibration for conversion to endotoxin or rFC-reactive substances, validation of performance or meeting compendia or the optical apparatus manufacturers' specifications, and measurement of the samples being analyzed.

The light impermeable testing apparatus containing optical property-modifying additives may be made from any suitable material. In another embodiment, portions of the light impermeable testing apparatus containing optical property-modifying additives may be coated with polymer materials, surface treatments, or coatings to meet compendia or the detection reagent manufacturers' specifications. In yet another embodiment, a portion of the light impermeable testing apparatus containing optical property-modifying additives may be coated with a static coating to reduce rFC reagent or standard loss. Another portion of the light impermeable testing apparatus containing optical property-modifying additives may be coated with a dynamic coating of the microplate wells to reduce rFC reagent or standards loss. The dynamic coating may also be mixed with standards or rFC reagents.

A portion of the light impermeable testing apparatus containing optical property-modifying additives may also be coated with additives to aid, or regulate, proper analysis and interactions of detection reagents or sample materials. Exemplary additives include, but are not limited to solubility aids, transport aids, and stabilizers.

In yet another embodiment, the light impermeable testing apparatus containing optical property-modifying additives may comprise mechanical barriers separate reagents to prevent interaction as they are being isolated in the light impermeable testing apparatus containing optical property-modifying additives or being stored long-term. The barriers may be insoluble and arranged such that they do not interfere with fluorescence measurements. Other barriers may be soluble to some extent so that they dissolve during measurement and do not interfere with it.

In yet another embodiment, the light impermeable testing apparatus containing optical property-modifying additives may be preloaded with standards and spikes made from control standard endotoxin (CSE) or reference standard endotoxin (RSE). The spikes may be stored as dried material so that they are at the correct concentration while not diluting or interfering with the sample being spiked.

The following example demonstrates an embodiment wherein rFC-reactive standards are preloaded onto the light impermeable testing apparatus containing optical property-modifying additives. The rFC-reactive standard range is shown in Table 1. The rFC-reactive standard range, however, may be different in other embodiments.

TABLE 1

| Range | Lowest | Mid-Range | Highest |
|---|---|---|---|
| (EU/mL) | (EU/mL) | (EU/mL) | (EU/mL) |
| 0.005-0.5 | 0.005 | 0.05 | 0.5 |
| 0.01-1 | 0.01 | 0.1 | 1 |
| 0.05-5 | 0.05 | 0.5 | 5 |
| 0.1-10 | 0.1 | 1 | 10 |
| 0.5-50 | 0.5 | 5 | 50 |

Table 2 is a description of the preloaded light impermeable testing apparatus containing optical property-modifying additives, wherein the light impermeable testing apparatus containing optical property-modifying additives has 96 portions. Column 1 indicates the portion of the light impermeable testing apparatus containing optical property-modifying additives. Column 2 indicates the sample that the operator must add to the light impermeable testing apparatus containing optical property-modifying additives. Each portion of the light impermeable testing apparatus containing optical property-modifying additives may be preloaded with a different quantity of rFC-reactive material, such that when reconstituted with 100 μL of BET water, the final concentration when exposed to rFC-reagents is as shown in Column 3. Column 4 is a description of the BET test that may be completed in each portion. The detection reagent is not shown in Table 2 as all 96 portions may be preloaded with the same amount of a detection reagent. Alternatively, the light impermeable testing apparatus containing optical property-modifying additives may not have any reagent, allowing the operator to add a reagent from a preferred supplier.

TABLE 2

| | | Endotoxin | |
|---|---|---|---|
| Well | Sample | Standard | Description |
| 1 | Water for BET | 0 | Negative Control (Blank) Replicate 1 |
| 2 | Water for BET | 0 | Negative Control (Blank) Replicate 2 |
| 3 | Water for BET | 0 | Negative Control (Blank) Replicate 3 |
| 4 | Water for BET | Lowest | Lowest Detection Range Calibration Standard Replicate 1 |
| 5 | Water for BET | Lowest | Lowest Detection Range Calibration Standard Replicate 2 |
| 6 | Water for BET | Lowest | Lowest Detection Range Calibration Standard Replicate 3 |
| 7 | Water for BET | Mid-Range | Mid-Range Calibration Standard Replicate 1 |
| 8 | Water for BET | Mid-Range | Mid-Range Calibration Standard Replicate 2 |
| 9 | Water for BET | Mid-Range | Mid-Range Calibration Standard Replicate 3 |
| 10 | Water for BET | Highest | Highest Detection Range Calibration Standard Replicate 1 |
| 11 | Water for BET | Highest | Highest Detection Range Calibration Standard Replicate 2 |
| 12 | Water for BET | Highest | Highest Detection Range Calibration Standard Replicate 3 |
| 13 | Sample A | 0 | Sample An Analysis Replicate 1 |
| 14 | Sample A | 0 | Sample An Analysis Replicate 2 |
| 15 | Sample A | Mid-Range | Positive Control Spike for Sample A Replicate 1 |
| 16 | Sample A | Mid-Range | Positive Control Spike for Sample A Replicate 2 |
| 17 | Sample B | 0 | Sample B Analysis Replicate 1 |
| 18 | Sample B | 0 | Sample B Analysis Replicate 2 |
| 19 | Sample B | Mid-Range | Positive Control Spike for Sample B Replicate 1 |
| 20 | Sample B | Mid-Range | Positive Control Spike for Sample B Replicate 2 |
| 21 | Sample C | 0 | Sample C Analysis Replicate 1 |
| 22 | Sample C | 0 | Sample C Analysis Replicate 2 |
| 23 | Sample C | Mid-Range | Positive Control Spike for Sample C Replicate 1 |
| 24 | Sample C | Mid-Range | Positive Control Spike for Sample C Replicate 2 |
| 25 | Sample D | 0 | Sample D Analysis Replicate 1 |
| 26 | Sample D | 0 | Sample D Analysis Replicate 2 |
| 27 | Sample D | Mid-Range | Positive Control Spike for Sample D Replicate 1 |
| 28 | Sample D | Mid-Range | Positive Control Spike for Sample D Replicate 2 |
| 29 | Sample E | 0 | Sample E Analysis Replicate 1 |
| 30 | Sample E | 0 | Sample E Analysis Replicate 2 |
| 31 | Sample E | Mid-Range | Positive Control Spike for Sample E Replicate 1 |
| 32 | Sample E | Mid-Range | Positive Control Spike for Sample E Replicate 2 |
| 33 | Sample F | 0 | Sample F Analysis Replicate 1 |
| 34 | Sample F | 0 | Sample F Analysis Replicate 2 |
| 35 | Sample F | Mid-Range | Positive Control Spike for Sample F Replicate 1 |
| 36 | Sample F | Mid-Range | Positive Control Spike for Sample F Replicate 2 |
| 37 | Sample G | 0 | Sample G Analysis Replicate 1 |
| 38 | Sample G | 0 | Sample G Analysis Replicate 2 |
| 39 | Sample G | Mid-Range | Positive Control Spike for Sample G Replicate 1 |
| 40 | Sample G | Mid-Range | Positive Control Spike for Sample G Replicate 2 |
| 41 | Sample H | 0 | Sample H Analysis Replicate 1 |
| 42 | Sample H | 0 | Sample H Analysis Replicate 2 |
| 43 | Sample H | Mid-Range | Positive Control Spike for Sample H Replicate 1 |
| 44 | Sample H | Mid-Range | Positive Control Spike for Sample H Replicate 2 |
| 45 | Sample I | 0 | Sample I Analysis Replicate 1 |
| 46 | Sample I | 0 | Sample \| Analysis Replicate 2 |
| 47 | Sample I | Mid-Range | Positive Control Spike for Sample I Replicate 1 |
| 48 | Sample I | Mid-Range | Positive Control Spike for Sample I Replicate 2 |
| 49 | Sample J | 0 | Sample J Analysis Replicate 1 |
| 50 | Sample J | 0 | Sample J Analysis Replicate 2 |
| 51 | Sample J | Mid-Range | Positive Control Spike for Sample J Replicate 1 |
| 52 | Sample J | Mid-Range | Positive Control Spike for Sample J Replicate 2 |
| 53 | Sample K | 0 | Sample K Analysis Replicate 1 |
| 54 | Sample K | 0 | Sample K Analysis Replicate 2 |
| 55 | Sample K | Mid-Range | Positive Control Spike for Sample K Replicate 1 |
| 56 | Sample K | Mid-Range | Positive Control Spike for Sample K Replicate 2 |
| 57 | Sample L | 0 | Sample L Analysis Replicate 1 |
| 58 | Sample L | 0 | Sample L Analysis Replicate 2 |
| 59 | Sample L | Mid-Range | Positive Control Spike for Sample L Replicate 1 |
| 60 | Sample L | Mid-Range | Positive Control Spike for Sample L Replicate 2 |
| 61 | Sample M | 0 | Sample M Analysis Replicate 1 |
| 62 | Sample M | 0 | Sample M Analysis Replicate 2 |
| 63 | Sample M | Mid-Range | Positive Control Spike for Sample M Replicate 1 |
| 64 | Sample M | Mid-Range | Positive Control Spike for Sample M Replicate 2 |
| 65 | Sample N | 0 | Sample N Analysis Replicate 1 |
| 66 | Sample N | 0 | Sample N Analysis Replicate 2 |
| 67 | Sample N | Mid-Range | Positive Control Spike for Sample N Replicate 1 |
| 68 | Sample N | Mid-Range | Positive Control Spike for Sample N Replicate 2 |
| 69 | Sample O | 0 | Sample O Analysis Replicate 1 |
| 70 | Sample O | 0 | Sample O Analysis Replicate 2 |
| 71 | Sample O | Mid-Range | Positive Control Spike for Sample O Replicate 1 |
| 72 | Sample O | Mid-Range | Positive Control Spike for Sample O Replicate 2 |
| 73 | Sample P | 0 | Sample P Analysis Replicate 1 |
| 74 | Sample P | 0 | Sample P Analysis Replicate 2 |
| 75 | Sample P | Mid-Range | Positive Control Spike for Sample P Replicate 1 |
| 76 | Sample P | Mid-Range | Positive Control Spike for Sample P Replicate 2 |
| 77 | Sample Q | 0 | Sample Q Analysis Replicate 1 |
| 78 | Sample Q | 0 | Sample Q Analysis Replicate 2 |
| 79 | Sample Q | Mid-Range | Positive Control Spike for Sample Q Replicate 1 |
| 80 | Sample Q | Mid-Range | Positive Control Spike for Sample Q Replicate 2 |
| 81 | Sample R | 0 | Sample R Analysis Replicate 1 |
| 82 | Sample R | 0 | Sample R Analysis Replicate 2 |
| 83 | Sample R | Mid-Range | Positive Control Spike for Sample R Replicate 1 |
| 84 | Sample R | Mid-Range | Positive Control Spike for Sample R Replicate 2 |
| 85 | Sample S | 0 | Sample S Analysis Replicate 1 |
| 86 | Sample S | 0 | Sample S Analysis Replicate 2 |
| 87 | Sample S | Mid-Range | Positive Control Spike for Sample S Replicate 1 |
| 88 | Sample S | Mid-Range | Positive Control Spike for Sample S Replicate 2 |
| 89 | Sample T | 0 | Sample T Analysis Replicate 1 |
| 90 | Sample T | 0 | Sample T Analysis Replicate 2 |
| 91 | Sample T | Mid-Range | Positive Control Spike for Sample T Replicate 1 |
| 92 | Sample T | Mid-Range | Positive Control Spike for Sample T Replicate 2 |
| 93 | Sample U | 0 | Sample U Analysis Replicate 1 |
| 94 | Sample U | 0 | Sample U Analysis Replicate 2 |
| 95 | Sample U | Mid-Range | Positive Control Spike for Sample U Replicate 1 |
| 96 | Sample U | Mid-Range | Positive Control Spike for Sample U Replicate 2 |

1. A preloaded testing apparatus for use in detecting rFC-reactive substances samples comprising:
a. At least one reaction well, but may contain a plurality of reaction wells, each one of the plurality of wells having at least one side well that defines the interior of the one of plurality of wells;
b. Light impermeable material forming at least the side of each of the wells to absorb light and prevent light from passing between adjacent wells or outside of the well
c. Wherein at least a portion of said preloaded testing apparatus has been preloaded with at least one rFC-reactive standard, or preloaded with at least one component of a detection reagent and at least one rFC-reactive standard, and wherein said rFC-reactive standard is present in a plurality of quantities and wherein each quantity is present on a separate portion of said preloaded testing apparatus.

2. The preloaded testing apparatus of claim 1, wherein at least a portion of said preloaded testing apparatus has a modified surface.

3. The preloaded testing apparatus of claim 2, wherein said modified surface is modified using plasma etching.

4. The preloaded testing apparatus of claim 2, wherein said modified surface is modified using at least one coating, wherein said coating is a static coating, dynamic coating, or combinations thereof.

5. The preloaded testing apparatus of claim 4, wherein at least one static coating is selected from the group consisting of polyethylene glycol (PEG), collagen, and combinations thereof.

6. The preloaded testing apparatus of claim 4, wherein at least one dynamic coating is selected from the group consisting of polyethylene glycol (PEG), sodium deoxycholate, and combinations thereof.

7. The preloaded testing apparatus of claim 1, further comprising at least one mechanical barrier between at least one of said separate portions.

8. The preloaded testing apparatus of claim 7, wherein at least one mechanical barrier is soluble.

9. The preloaded testing apparatus of claim 1, wherein said preloaded testing apparatus further comprises a portion identification mechanism.

10. The preloaded testing apparatus of claim 1, wherein said preloaded testing apparatus is a microplate.

11. The preloaded testing apparatus of claim 1, wherein said preloaded testing apparatus further comprises a barrier material to protect said preloaded testing apparatus from environment exposure and surface contamination.

12. A method for measuring a Factor C reactive substance in a sample, said method comprising:
a. contacting said sample with a preloaded testing apparatus wherein at least a portion of said preloaded testing apparatus has been preloaded with at least one rFC-reactive standard, or preloaded with at least one test reagent comprising a rFC-detection reagent and at least one rFC-reactive standard, and wherein said rFC-reactive standard is present in a plurality of volumes and wherein each volume is present on a separate portion of said testing apparatus, thereby making a prepared sample; and
b. measuring a fluorescence of said prepared sample.

13. The method of claim 12, wherein at least a portion of said preloaded light impermeable testing apparatus containing optical property-modifying additives has a modified surface.

14. The method of claim 13, wherein said modified surface is modified using plasma etching.

15. The method of claim 13, wherein said modified surface is modified using at least one coating, wherein said coating is a static coating, dynamic coating, or combinations thereof.

16. The method of claim 15, wherein at least one static coating is selected from the group consisting of polyethylene glycol (PEG), collagen, and combinations thereof.

17. The method of claim 15, wherein at least one dynamic coating is selected from the group consisting of polyethylene glycol (PEG), sodium deoxycholate, and combinations thereof.

18. The method of claim 12, wherein said plurality of rFC-reactive standard concentrations is used to generate a standard curve.

19. The method of claim 12, wherein said preloaded light impermeable testing apparatus containing optical property-modifying additives is a microplate.

20. The method of claim 12, wherein said preloaded light impermeable testing apparatus containing optical property-modifying additives further comprises a barrier material to protect said preloaded light impermeable testing apparatus containing optical property-modifying additives from environmental exposure and surface contamination.

21. A method for depositing at least one detection reagent on a light impermeable testing apparatus containing optical property-modifying additives, said method comprising:
a. taking a light impermeable testing apparatus containing optical property-modifying additives having a well array comprising a plurality of wells, wherein each well has at least one optical window surface and a plurality of non-optical window surfaces;
b. placing a first liquid solution having at least one detection reagent therein on a first non-optical window surface of at least one well;
c. drying said first liquid solution on said first non-optical window surface, thereby depositing said detection reagent on said first non-optical window surface and forming a preloaded light impermeable testing apparatus containing optical property-modifying additives;
d. placing a second liquid solution having at least one rFC-reactive standard therein on a second non-optical window surface of at least one well, and wherein said rFC-reactive standard is present in a plurality of concentrations; and
e. drying said second liquid solution on said second non-optical window surface, thereby depositing said rFC-reactive standard on said second non-optical window surface.

22. A method for depositing at least one detection reagent on a light impermeable testing apparatus containing optical property-modifying additives, said method comprising:
a. taking a light impermeable testing apparatus containing optical property-modifying additives having a well array comprising a plurality of wells, wherein each well has at least one optical window surface and a plurality of non-optical window surfaces;
b. placing a first liquid solution having at least one detection reagent therein on a first non-optical window surface of at least one well;
depositing said detection reagent on said first non-optical window surface and forming a preloaded light impermeable testing apparatus containing optical property-modifying additives;
d. placing a second liquid solution having at least one rFC-reactive standard therein on a second non-optical window surface of at least one well, and wherein said rFC-reactive standard is present in a plurality of concentrations; and
e. drying said second liquid solution on said second non-optical window surface, thereby depositing said rFC-reactive standard on said second non-optical window surface.

23. The method of claim 21, wherein said preloaded light impermeable testing apparatus containing optical property-modifying additives is a microplate.

24. The method of claim 21, wherein said method further comprises covering said preloaded light impermeable testing apparatus containing optical property-modifying additives with a barrier material after said drying step to protect said preloaded light impermeable testing apparatus containing optical property-modifying additives from environmental exposure and surface contamination.

25. A method for depositing at least one detection reagent on a light impermeable testing apparatus containing optical property-modifying additives, said method comprising:
a. taking a light impermeable testing apparatus containing optical property-modifying additives having a well array comprising a plurality of wells, wherein each well has at least one optical window surface and a plurality of non-optical window surfaces;
b. placing a first liquid solution having at least one detection reagent therein on a first non-optical window surface of at least one well;
depositing said detection reagent on said first non-optical window surface and forming a preloaded light impermeable testing apparatus containing optical property-modifying additives;
d. placing a second liquid solution having at least one rFC-reactive standard therein on a second non-optical window surface of at least one well, and wherein said rFC-reactive standard is present in a plurality of volumes; and
e. drying said second liquid solution on said second non-optical window surface, thereby depositing said rFC-reactive standard on said second non-optical window surface.

26. The method of claim 25, wherein said preloaded light impermeable testing apparatus containing optical property-modifying additives is a microplate.

27. The method of claim 25, wherein said method further comprises covering said preloaded light impermeable testing apparatus containing optical property-modifying additives with a barrier material after said drying step to protect said preloaded light impermeable testing apparatus containing optical property-modifying additives from environmental exposure and surface contamination.
